(19) United States
(12) Patent Application Publication
(10) Pub. No.: US 2013/0209582 A1
(43) Pub. Date: Aug. 15, 2013

(54) HYDROGEN IODIDE-CONTAINING HEALTH FOOD AND HYDROGEN IODIDE-CONTAINING DRUG, AND METHOD FOR PRODUCING SAME

(71) Applicant: Kazuyoshi Sato, Tokyo (JP)

(72) Inventor: Kazuyoshi Sato, Tokyo (JP)

(73) Assignee: Kazuyoshi SATO, Tokyo (JP)

(21) Appl. No.: 13/823,363

(22) PCT Filed: Sep. 27, 2010
(86) PCT No.: PCT/JP2010/066720

§ 371 Date: Apr. 9, 2013

(51) Int. Cl. A61K 33/18; A23L 1/304

(52) U.S. Cl. CPC A61K 33/18; A23L 1/304
USPC 424/667; 423/481

(57) ABSTRACT

To propose a health food or a drug, which comprises an iodine compound as a substitute for organic iodine and can exert a similar effect as organic iodine. To propose a method for producing a liquid to be used as a starting material for the aforesaid health food or drug. A health food and a drug each containing hydrogen iodide as the active ingredient. A method for producing a hydrogen iodide-containing solution, which comprises: an alkaline ion water preparation step for preparing alkaline ion water containing hydrogen peroxide; a sodium hydroxide production step for adding sodium to the alkaline ion water and thus producing sodium hydroxide; and a hydrogen iodide production step for, after the sodium hydroxide production step, adding iodine to the alkaline ion water and thus producing hydrogen iodide.

### FIELD OF THE INVENTION

The present invention relates to health foods for promotion of health and pharmaceutical products for prevention and treatment of diseases.

### BACKGROUND ART

Conventionally, iodine has been used as an ingredient for antiseptic or hemostatic. However, iodine is a highly responsive substance, and when administering it to humans at high doses, it affects the human body. Therefore, maximum daily intake of iodine is limited to 100 mg.

In Japanese Unexamined Patent Application Publication No. 2004-315470, pharmaceutical preparation containing sodium iodide, a weak alkaline solution containing sodium iodide, is disclosed. This pharmaceutical preparation offers advantages in anti-AIDS with extremely low side effects such as iodism, even when the daily intake of iodine exceeds 100 mg. However, in this pharmaceutical preparation, iodine is combined with sodium, so that the iodine is affected by the sodium and is not efficiently absorbed into the body.

In Japanese Unexamined Patent Application Publication No. 2008-143808, a liquid preparation, a weak alkaline solution containing dispersed organic iodine, is disclosed. Similar to the pharmaceutical preparation containing sodium iodide, this liquid preparation containing organic iodine offers advantages in prevention and treatment of cancer or AIDS with extremely low side effects such as iodism, even when the daily intake of iodine exceeds 100 mg. Moreover, this liquid preparation allows the iodine to be efficiently absorbed into the body in contrast with the pharmaceutical preparation of Japanese Unexamined Patent Application Publication No. 2004-315470, thereby producing a high therapeutic effect.

### RELATED ART DOCUMENTS

Patent Document 1: Japanese Unexamined Patent Application Publication No. 2004-315470

Patent Document 2: Japanese Unexamined Patent Application Publication No. 2008-143808

### DISCLOSURE OF THE INVENTION

### Problems that the Invention tries to Solve

An aspect of the present invention provides a method for producing a health food or a pharmaceutical product, which contains iodine compound other than organic iodine, and offers advantages in health promotion or treatment of disease with extremely low side effects such as iodism even when the daily intake of iodine exceeds 100 mg. Moreover, the present invention provides a method for producing a solution used as a raw material of health food etc.

### Means for Solving the Problems

The present invention is based upon the discovery by the inventor of this invention that when a substance containing hydrogen iodide was taken into the body, the substance was effective in health promotion and prevention and treatment of cancer or AIDS. Moreover, a method for efficiently producing a solution containing the hydrogen iodide is provided.

### BRIEF DESCRIPTION OF THE DRAWINGS

FIG. 1 is a flowchart showing production of hydrogen iodide-containing water.

### DETAILED DESCRIPTION OF THE INVENTION

### Outline

An aspect of the present invention provides a health food or a pharmaceutical product, which contains hydrogen iodide as an active ingredient. When iodine atoms in the hydrogen iodide are taken into a body, it is bound to protein, and forms a substance having a negative charge. This substance has effects of activating normal cells and destroying cancer cells and the AIDS virus. Therefore, the substance containing hydrogen iodide as an active ingredient acts as a health food or a drug for prevention or treatment of cancer or AIDS.

### Ingredient and Effect

The health food or pharmaceutical product of the present invention contains hydrogen iodide as the active ingredient. Here, hydrogen iodide is an inorganic compound having iodine and hydrogen, and is soluble in water. Hydrogen iodide is gas at ordinary temperature.

When the hydrogen iodide is taken into a body, hydrogen ions are separated due to chlorine in the body, and forms iodine atoms (I₂⁻) having a negative charge. These iodine atoms having a negative charge are bound to protein in the thyroid gland. The protein bound to the iodine atom (hereinafter, referred to as colloidal iodine) has a negative charge, and moves while repelling with other colloidal iodine.

The colloidal iodine can sublimate and remove extra compounds contained in minerals such as ion, magnesium and manganese. Therefore, the colloidal iodine can change the minerals to pure minerals. Therefore, the normal cells can receive normal electrons from pure minerals, thereby acting stably. Moreover, the electron of the iodine atom can activate normal cells. Therefore, health food containing the hydrogen iodide as the active ingredient works effectively.

Moreover, the diameter of the colloidal iodine is 10⁻⁸ to 10⁻⁵ cm, and therefore, can be ingested by a microorganism and a cancer cell. Cancer cells ingest the protein and grow. Therefore, similar to other proteins, the colloidal iodine is ingested into the cancer cell. When the colloidal iodine is ingested into the cancer cell, the iodine atom in the colloidal iodine emits electrons, so that destruction of nuclear membrane of the cancer cell is caused, thereby destroying the cancer cell. The iodine atom, which has emitted the electron, forms iodine salt (I₂) and is eliminated from the body. Therefore, pharmaceutical products containing hydrogen iodide as an active ingredient effectively works for prevention or treatment of cancer.

Furthermore, when injecting hydrogen iodide into the blood, the hydrogen ion is bound to protein in the blood and forms the colloidal iodine. AIDS virus (HIV) penetrates into the blood of the body, and ingests the protein in the blood. Therefore, similar to other proteins, the colloidal iodine is ingested into the AIDS virus. When the colloidal iodine is ingested into the AIDS virus, the iodine atom in the colloidal iodine emits electrons, so that destruction of cell nuclear membrane of the AIDS virus is caused, thereby destroying the AIDS virus. The iodine atom, which has emitted the electron, forms iodine salt (I₂) and is eliminated from the body. Therefore, the pharmaceutical product containing hydrogen iodide as an active ingredient effectively works for prevention or treatment of the AIDS.

Moreover, health food or pharmaceutical products containing hydrogen iodide as the active ingredient may be liquid form or solid form. Note that even in the case of the health food or pharmaceutical products in the solid form, hydrogen iodide exists in water solution at normal temperature, so that it is required to preserve this liquid ingredient without being vaporized. The content of the hydrogen iodide may be 1-10 wt. % of the liquid ingredient.

Moreover, health food or a pharmaceutical product may contain ingredients such as various amino acids, glucose, vitamin supplements, stabilizing agents, soothing agents, and coloring agents other than the hydrogen iodide.

### Dosage

When taking hydrogen iodide into the body, the hydrogen iodide reaches to the thyroid gland within 30 minutes through gastrointestinal absorption. Since the hydrogen iodide is retained in the thyroid gland for about two hours, it is preferable to take the health food or the pharmaceutical product every two hours in a serious case. Moreover, it is preferable to take them not in large quantities at one time, but in small quantities frequently on an empty stomach.

Moreover, it is preferable to take health food or a pharmaceutical product basically by oral ingestion except the cases of AIDS and leukemia. In the cases of AIDS and leukemia, it is preferable to take them by injection. In the case of terminal cancer, it is preferable to take them by injection along with the oral ingestion.

### Production Method

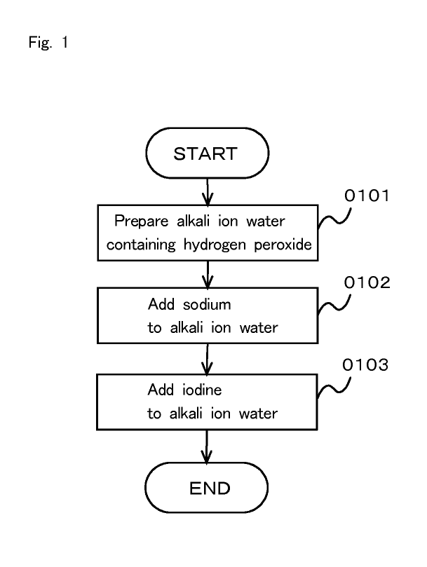

A method for producing water containing the hydrogen iodide used in health food or the pharmaceutical product of the present invention has the following steps as shown in FIG. 1. At the outset, in step S0101, alkali ion water containing hydrogen peroxide is prepared (step of preparing alkali ion water). Subsequently, in step of S0102, sodium hydroxide is produced by adding sodium to the alkali ion water (step of producing sodium hydroxide). Subsequently, in step S0103, dispersed hydrogen iodide is produced by adding iodine to the alkali ion water after producing the sodium hydroxide (step of producing dispersed hydrogen iodide).

The alkali ion water containing hydrogen peroxide prepared in the ‘step of preparing alkali ion water’ can be produced, for example, by bombarding it with raw water with piezoelectric ceramics.

When bombarding the raw water (H₂O) with the piezoelectric ceramics, partial water is separated into ions, thereby producing hydrogen ion (H⁺) and hydroxide ion (OH⁻). Moreover, the piezoelectric ceramics emit electron shower upon bombarding with the raw water, and provides the electron to partial oxygen molecule (O₂) in the raw water, thereby producing oxide anion (O₂⁻). The oxide anion is an active oxide with high responsiveness, so that it reacts to the hydrogen ion (H⁺) in the raw water, thereby producing a slight amount of hydrogen peroxide (H₂O₂). This hydrogen peroxide exerts a bactericidal effect in the body.

Moreover, it is possible to dissolve hydrogen peroxide produced by other chemical reactions with the alkali ion water. The pH of the alkali ion water may be 9-9.5.

In the ‘step of producing sodium hydroxide’, the hydroxide ion (OH⁻) having higher concentration than the hydrogen ion (H⁺) and sodium (Na) as electrolyte are bound, thereby producing sodium hydroxide (NaOH). It is preferable to determine the amount of the sodium according to the amount of the hydroxide ion (OH⁻), and the amount of the sodium hydroxide in the liquid may be 1-5 wt. %. Since the valence shell of the hydroxide is full due to the above reaction, the hydrogen ion becomes more reactive.

In the ‘step of producing dispersed hydrogen iodide’, the above high-reactive hydrogen ion and the iodine are bound, thereby producing hydrogen iodide (2HI). It is preferable to determine the amount of the iodine according to the amount of the hydrogen ion, and the amount of the hydrogen iodide in the liquid may be 1-10 wt. %. The produced hydrogen iodide (2HI) is electrical charged, so that molecules repel each other. Therefore, the hydrogen iodide exists in dispersion state without binding to each other, thereby not causing toxicity.

### Effect

Health food or pharmaceutical products of the present invention containing the hydrogen iodide as the active ingredient work as a health food or a drug for prevention or treatment of cancer or AIDS.

### Example 1

### Production Example

Solution Containing Hydrogen Iodide

Sodium 2.0 g is added to a 9.5 pH alkali ion water 96 g, and vigorously stirred. Then, iodine 2.0 g is added, thereby producing solution containing hydrogen iodide.

### Details of Clinical Research

A 20 ml solution produced by doubling dilution of the 10 ml solution produced as the above was administered orally four to eight times a day on an empty stomach, and examinations such as blood examination, tumor marker examination and CT scan are carried out.

### Example of Clinical Research 1

43-Year-Old Man having Lung Cancer Spreading to the Brain.

(Before Starting Administration)

The case of an abenocancer which has come back after the surgery and metastatic cancers spread to the lung at several portions and to the brain at a portion which was 3 cm in diameter. The patient had a stuffy head due to the metastatic cancer in the brain. An anticancer drug was not effective, and this case was diagnosed as a terminal cancer.

(After Starting Administration)

Administration of the solution was started, tumor shrinkage was found in MRI brain scan two months later, and the focus was not found in MRI brain scan three months later. Although the tumor marker CEA was 149 before administration, after administration, it dropped to 68 one month later, and further dropped to 31 two months later. Finally, the tumor was not found, and therefore, achieved a remission.

### Example of Clinical Research 2

61-Year-Old Woman having Blood Vessel Leiomyosarcoma

(Before Starting Administration)

The case of blood vessel leiomyosarcoma at descending aorta Although the patient underwent surgery to remove the blood vessel and blood vessel prosthesis implantation, many metastatic focuses were found in lung and liver two years later. Further operation was impossible and anticancer drug and radiation treatment were not effective. Before starting administration, 200 metastatic tumors were found in the lung, liver and lymph node etc.

(After Starting Administration)

Administration of the solution was started, and the patient felt recovery of physical strength two months later. After that, it was found in CT scan that the tumor has not disappeared, but its growth has stopped. The patient has sustained her physical condition for two years since starting administration.

### Example of Clinical Research 3

52-Year-Old Woman having Breast Cancer

(Before Starting Administration)

The patient found a lump in her breast and a malignant tumor of 2 cm in a diameter was found in the right breast by a check. The patient was diagnosed with breast cancer at stage 2B, and declined treatments by operation, radiation treatment, and anticancer drug.

(After Starting Administration)

Administration of the solution was started, and the patient felt reduction of the lump two months later. The tumor in the breast was not found in an examination two months after starting administration, and three months later, it was diagnosed that the cancer was cured. After that, recurrence of the cancer has not been found for two years.

### Example of Clinical Research 4

65-Year-Old Man having Colorectal Cancer

(Before Starting Administration)

The patient underwent surgery to remove colorectal cancer and anticancer drug treatment. However, two years later, metastatic cancer was found in the lung.

(After Starting Administration)

Administration of the solution was started, the patient's appetite was improved, and other side effects were not shown. Reduction of the liver metastatic tumor marker was shown in an examination six months after starting administration.

### Example of Clinical Research 5

60-Year-Old Man having Gallbladder Cancer

(Before Starting Administration)

The patient became jaundiced and was diagnosed by ultrasound examination that surgery was necessary. After that, although the patient underwent pancreatoduodenectomy, metastatic tumor was found in the lymph node.

(After Starting Administration)

Administration of the solution was started along with the anticancer drug treatment, and increase of tumor marker and hepatic dysfunction were not shown six months after starting administration.

### Example of Clinical Research 6

54-Year-Old Man having Gastric Cancer

(Before Starting Administration)

The patient was diagnosed with IIc type gastric cancer, and gastrectomy by opening the abdomen was scheduled. Since there was time until the date of the surgery, the patient started administration of the solution.

(After Starting Administration)

Administration of the solution was started, and reduction of the tumor was found by an endoscopic examination eight weeks later. The patient underwent endoscopic mucosal resection and is making good progress.

What is claimed is:

1. A health food, comprising:
hydrogen iodide as an active ingredient.

2. A liquid health food, comprising:
hydrogen iodide as an active ingredient.

3. The liquid health food according to claim 2,
wherein acid level of the health food indicates alkaline property.

4. A pharmaceutical product, comprising:
hydrogen iodide as an active ingredient.

5. A liquid pharmaceutical product, comprising:
hydrogen iodide as an active ingredient.

6. The liquid pharmaceutical product according to claim 5,
wherein acid level of the pharmaceutical product indicates alkaline property.

7. A method for producing hydrogen iodide-containing solution, comprising:
preparing alkali ion water containing hydrogen peroxide;
producing sodium hydroxide by adding sodium to the alkali ion water; and
producing hydrogen iodide by adding iodine to the alkali ion water after producing the sodium hydroxide.
